(19) United States
(12) Patent Application Publication
(10) Pub. No.: US 2024/0194308 A1
(43) Pub. Date: Jun. 13, 2024

(54) MAPPING OF A DATABASE FIELD IN AN ELECTRONIC HEALTH RECORD TO A QUESTION ON A CLINICAL TRIAL CASE REPORT FORM (CRF) USING THE ELECTRONIC DATA DOCUMENT (EDD)

(71) Applicant: Akyrian Systems LLC, New York, NY (US)

(72) Inventors: Edward Ikeguchi, New York, NY (US); Peter T. Jackson, Brooklyn, NY (US)

(73) Assignee: Akyrian Systems LLC, New York, NY (US)

(21) Appl. No.: 18/534,142

(22) Filed: Dec. 8, 2023

Related U.S. Application Data
(60) Provisional application No. 63/431,517, filed on Dec. 9, 2022.

(51) Int. Cl. G16H 10/60; G16H 10/20

(52) U.S. Cl. CPC G16H 10/60; G16H 10/20

(57) ABSTRACT

A system and method that allows the usage of an electronic data document as a means with which to map the database fields in an electronic health record to a question on a clinical report form using an application programming interface. The electronic data document can associate a clinical report form question with a source capture from an electronic health record by correlating it via a snippet to the content on the source capture that can then be confirmed by a user. Positive matches can lead to a streamlined process where the same question on the clinical report form during a future visit with the patient results in the electronic data document system automatically pulling the answer from the electronic health record via the application programming interface using the previously defined field identifier and populating it into the case report form.

### CROSS REFERENCE TO RELATED APPLICATIONS

This application claims the benefit of U.S. Provisional Patent Application Ser. No. 63/431,517, filed Dec. 9, 2022, to Ikeguchi, et al., titled “Mapping of a Database Field in an Electronic Health Record to a Question on a Clinical Trial Case Report Form (CRF) Using the Electronic Data Document (EDD)”, currently pending, the contents thereof being incorporated herein by reference.

### BACKGROUND OF THE INVENTION

In U.S. Pat. Nos. 10,706,958, 10,811,122, 11,562,810, 11,562,811, U.S. patent application Ser. No. 18/084,969, and U.S. patent application Ser. No. 18/038,862, the contents of each being incorporated herein by reference, the inventors of the present invention previously described the concept of an Electronic Data Documents (EDD) whereby one or more questions from a clinical trial questionnaire, i.e.—a Case Report Form (CRF), typically used to collect information from clinical trial subjects in a clinical trial according to the desired data to be collected in support of the clinical trial, is combined with image information from a source document (SD), typically an electronic medical/health record (EHR) or other document storing medical information related to the clinical trial subject but collected outside of the clinical trial, either historically or contemporaneously with the data collection in the clinical trial, i.e.—a Source Capture (SC).

These applications further described the creation of a source capture (SC) from an EHR page whereby a screen capture of an EHR page results in a picture (i.e.—image) of the original EHR page (so-called “Source Document”). The SC may be created by the site user (typically a doctor, nurse or other clinical research personnel) using the EDD software system. Once a SC is created, they then have the ability to generate a snippet, which is a delineated subsection of the SC containing the answer to a CRF question as determined by the site user or other. Once defined, the EDD system can associate the snippet with a question on a CRF. A process has also been described where the snippet can be converted from a picture to alphanumeric values using optical character recognition (OCR) which is used as a computer-generated suggestion for data entry.

These applications further describe a process where the system performs OCR on the entirety of an EHR page and uses computer vision and machine learning to automatically identify sections of the EHR page that contain the answer to a question on a CRF.

It is also well known that there are standardized computer programming interfaces or application programming interface (API) data models that allow parties external to an EHR system to programmatically access medical records in a secure, privacy-compliant fashion. One such prominent API standard is the FHIR (Fast Healthcare Interoperability Resources) standard, which defines how healthcare information can be exchanged between different computer systems.

Despite the availability of such infrastructure, the inventors of the present invention have recognized that these standards have been slow to be operationalized in clinical trials because the mapping of data points from a hospital database to a clinical database is tedious and time consuming. Furthermore, the large number of manufacturers of EHR systems paired with their highly customizable nature has led to tremendous heterogeneity between doctor's offices, including those using the same EHRs, thus leading to limitations in scale for usage in clinical trials.

### SUMMARY OF THE INVENTION

In accordance with one or more embodiments described with the present invention, a system and method are provided that allows the usage of the EDD as a means with which to map the database fields in an EHR to a question on a CRF. The value of this invention is that the data from a hospital EHR system will be mapped to specific clinical trial questions such that data acquisition can occur in the most seamless, secure, and time-efficient way.

In an embodiment of the invention, the EDD preferably associates a CRF question with a SC when it is correlated via a snippet to the content on the SC and confirmed by the user. This creates a snippet to CRF-question pair. The invention according to an embodiment further preferably adds steps to take the information in the snippet and seek a match in the EHR record for the same patient via the API integration. The system further uses the API connectivity to the EHR system to narrow the possible matches using other parameters available to the EDD system including: MRN, visit date, the name (i.e.—label) of a field or variable within the EHR (i.e. “Heart rate”), the value of a field or variable within the EHR (i.e.—74 beats per minute).

The same data is extracted from the EHR via two methodologies: 1) an SC/snippet derived via the EDD, and 2) an API message, serving as a bridge, allowing the EDD system to ultimately discover the data field's unique identifier (i.e.—a unique identifier for a database variable) in the EHR by using the API. The EDD system preferably further provides a UI to present the site user with results of any correlations between their snippets to data points within the EHR system discovered via the API integration, allowing the users to confirm a match of the data extracted by each method.

In some cases where multiple similar matches are discovered, the UI also provides the site user with a list of possible matches discovered in the EHR via the API integration to choose from. Users are also able to deny any matches and continue via the EDD (i.e.—snippet) method.

Positive matches lead to a streamlined process where the same question on the CRF during a future encounter/visit with the patient results in the EDD system automatically pulling the answer from the EHR via the API using the previously defined field identifier and populating it into the CRF. The system preferably develops greater accuracy each time the site user captures additional snippets, functioning as a supervised learning or training process by which the system continually improves and refines these mappings in response to users creating new snippets of the same questions from one patient visit to the next. Such learning or training may be performed by any known AI or machine learning processes.

The present invention provides a method for mapping a database field having data elements in an electronic health record to a question on a clinical trial case report form (CRF), including extracting an image of a source document included in the electronic health record and creating one or more snippets, wherein the one or more snippets includes data elements, selecting one or more of the snippets, recognizing one or more data elements from the one or more snippets, inputting one or more of the recognized data elements into an Application Programming Interface (API), creating one or more search terms using the one or more recognized data elements, searching an Electronic Health Record (EHR) using the API for one or more search terms, identifying one or more data elements in the EHR as corresponding to the one or more search terms, and presenting the one or more identified elements to a user.

The present invention also provides a computerized system for mapping a database field having data elements in an electronic health record to a question on a clinical trial case report form (CRF), including, at least one processor having a snippet tool extracting an image of a source document included in an electronic health record and creating one or more snippets, wherein the one or more snippets includes data elements, an Application Programming Interface (API), wherein one or more of the data elements are input into the Application Programming Interface (API) as a search term, and the at least one processor searching an Electronic Health Record (EHR) using the API for one or more search terms and identifying one or more data elements in the EHR as corresponding to the one or more search terms and presenting the one or more identified elements to a user.

The present invention also provides a computer processor configured to implement a method for mapping a database field having data elements in an electronic health record to a question on a clinical trial case report form (CRF) including extracting an image of a source document included in the electronic health record and creating one or more snippets, wherein the one or more snippets includes data elements, selecting one or more of the snippets, recognizing one or more data elements from the one or more snippets, inputting one or more of the recognized data elements into an Application Programming Interface (API), creating one or more search terms using the one or more recognized data elements, searching an Electronic Health Record (EHR) using the API for one or more search terms, identifying one or more data elements in the EHR as corresponding to the one or more search terms, and presenting the one or more identified elements to a user.

### BRIEF DESCRIPTION OF THE DRAWINGS

Further objects, features and advantages of the invention will become apparent from the following detailed description taken in conjunction with the accompanying figure showing an illustrative embodiment of the invention, in which:

FIG. 1 shows a flowchart of an exemplary embodiment of the present invention; and

FIG. 2 shows a flowchart of an exemplary embodiment of learned mappings used to extrapolate finding new matches.

### DETAILED DESCRIPTION

The inventors of the present invention have determined that at the current time, because clinical trial data is collected into electronic data capture (EDC) systems whereby transcription (i.e., the act of writing information in the form of glyphs, letters, or the like, in order to provide information in response to a question) is the means with which information is manually typed or keyed into a computer, the resulting data is a mosaic of incongruous values from hundreds of different hospitals and laboratories. Dedicated manual and software driven processes exist to convert values entered in one set of units into another using math.

As noted above, one key differentiator of the EDD compared to more traditional EDC is the usage of image data taken directly from source documents, i.e.—images of hospital records, and a mechanism to transfer data from the SD to the clinical database without the need for transcription. Since the SC is an image taken directly from the SD, the EDD eliminates the need to perform source document verification (SDV) whereby, for current processes using EDC, a reconciliation is performed between the SD and the clinical database as a manual and laborious quality control process to assure that the transcription process did not lead to erroneous data entry. Another advantage of the EDD is that it is contemplated to apply modern technologies as part of the system and method to interpret the contents of an image or SC. These modern technologies may include systems such as optical character recognition (OCR), robotic process automation (RPA), artificial intelligence (AI), generative AI processes, large language models (LLM), machine learning (ML), computer vision (CV), data object model (DOM) detection, natural language processing (NLP), among others. This gives the EDD a distinct advantage over EDC because data does not require transcription to move from one system (the SD in the EHR) to another (clinical database).

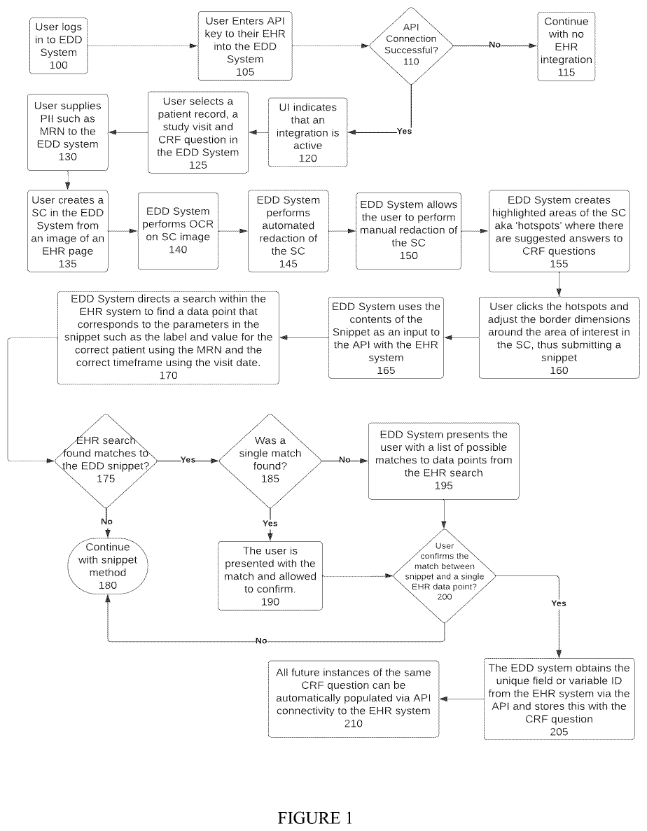

Referring to FIG. 1, an embodiment of the invention is shown. In FIG. 1, at a first step 100 a user first logs into an EDD system and at step 105 enters API key to their EHR into the EDD system. The EDD software preferably comprises a configuration page allowing a user to define any integrations with external systems using an API. Once a user is able to supply an API key and authenticate the connection, the EDD software will be integrated and connected to the EHR system for a particular site. The UI will indicate that a connection has been established. Processing then continues at step 110 where it is enquired whether the API connection was successful. If this enquiry at step 110 is answered in the negative, and it is therefore determined that the API connection was not successful, processing continues at step 115 without using any EHR integration.

If, on the other hand, the query at step 110 is answered in the affirmative, and it is therefore determined that the API connection was successful, processing instead continues at step 120 where the UI presented to the user indicates that an EHR integration is active. Next, at step 125 the user first either selects an existing patient, or adds a new one, and then defines or selects a study visit and a CRF study question in the EDD system. Processing continues at step 130 where the user supplies requested personally identifiable healthcare information, such as the Medical Record Number (MNR) to the EDD system. The EDD system may preferably ask the site user to input information that will identify the patient in the clinical trial. Patient specific information can include the patient's medical record number (MRN), the patient's name plus their date of birth, or other commonly used or agreed upon patient identifying information. The date of the patient's visit to the hospital or clinic can also be supplied to the EDD system by the site user.

Processing then continues at step 135 where the user creates a source capture (SC) in the EDD system from an image of an EHR page. Thus, the site user preferably opens their EHR system, navigates to the correct patient and page containing relevant medical information, and takes a screen capture. This is the created SC. Next, at step 140, the EDD system performs an OCR process on the created SC image. The system automatically scans via OCR the contents of the image and at step 145 automatically redacts any PII, including the information supplied by the user as described in the above-referenced applications incorporated herein by reference. In particular, when specific elements of the PII are identified, the system will replace the section of PII in the SC image with a watermark that is a permanent opaque square to fully obfuscate the underlying image of any text, but also replace the underlying text with a label for the underlying content such as “MRN” covering the patient's MRN or the patient's study specific identifier (ABC123) in place of their name. This is the process of automated redaction. Thus, information is not merely removed, but an indication is put in its place as to, perhaps, the type of data that was removed. Additionally, date of redaction, identity or redactor, and the like may be provided in accordance with such automated redaction. Additionally, at step 150, the EDD system also permits a user to perform additional manual redaction of the captured SC. Thus, in addition to the automated process of redaction, the system allows the user to manually click and drag with their mouse to draw opacities over other areas of the image they wish to redact.

After the redaction process is complete, processing continues at step 155 where the EDD system creates highlighted (or otherwise identified or designated) areas of the SC. These highlighted areas are referred to as “hotspots” and originate from key words that are configured into the EDD system during study setup, where a designated user with configuration privileges inputs search terms that would be discoverable on a SC. The system can handle multiple words for a single question. For example, the CRF question “What is the patient's temperature?” might be configured in the EDD backend with search terms such as “temperature”, “temp”, “Fahrenheit”, “Celsius”, etc. During the data acquisition process, the EDD system cross references textual content obtained during the OCR of the SC image against search terms and adds visually distinct areas to the SC as overlays that can be clicked the by the site user. Upon clicking, the dimensions of the hotspot can be adjusted to include not just the discovered label (i.e., the search term), but also the result and other metainformation in the vicinity (i.e.—units, normal reference ranges, etc.). This process of adjusting the hotspot ultimately redefines and confirms the borders of the desired information on the SC and, upon submission to the EDD system, is then referred to as the snippet. Note that the process of adjusting the dimensions of the hotspot to include other information can also be performed automatically by the EDD system through the use of adjunctive computer technologies such as computer vision and the use of heatmaps, templates, generative AI, NLP, and machine learning. The system therefore presents the site user with a UI that automatically suggests areas where answers to CRF questions are located as indicated as hotspots. Processing continues at step 160 where the user selects a hotspot by clicking the hotspot, or by other method of selection, and is then able to adjust the border dimensions around the area of interest in the SC, thus submitting a snippet. The site user can therefore use their mouse to click a hotspot to adjust the dimensions of the square surrounding an area of the EHR where an answer to a CRF question exists. Once confirmed this is then called a snippet, which is a circumscribed area of the SC that depicts the response to the CRF question and, optionally, the label of the question in the image. Once the user is satisfied with the dimensions of the snippet, the snippet can be submitted to the system for further processing. The system will then associate the finalized snippet with the CRF question and attach the source capture image and the snippet image to the data point's audit trail. The audit trail will allow a user to view the evolution of a datapoint in the system including image data such as the SC and snippet which serve as supporting evidence for any information accrued in the database, and any changes or other processing that has occurred to the datapoint or supporting evidence.

Processing then continues at step 165 where the EDD system preferably uses the contents of the snippet as an input to the API with the EHR system. At next step 170 the EDD system preferably directs a search within the EHR system to find a data point that corresponds to the parameters in the snippet such as the label and value for the correct patient using the MRN and the correct timeframe using the visit date. The system preferably obtains the OCR results from the snippet and cross-references the information from the snippet in a search through the patient's EHR via the API integration, with specific parameters such as the MRN, visit date, field label, and field value, and the like. At step 175 it is then queried whether the EHR search in step 170 found one or more matches to the EDD snippet. If the enquiry at step 175 is answered in the negative, and it is therefore determined that no matches are found, processing continues at step 180 using the snippet method (i.e. does not employ the mapping process in accordance with this embodiment of the present invention) where the information from the snippet is converted to alphanumeric characters (i.e. text or numeric characters) using OCR applied to the snippet image and entered into the CRF as set forth above in one or more of the applications incorporated herein by reference.

If, on the other hand, the enquiry at step 175 is answered in the affirmative, and it is therefore determined that one or more matches were found, processing continues at step 185 where it is further enquired whether single match was found. If the enquiry is answered in the affirmative, and it is therefore determined that a single match was found, the user is presented with the match and invited to confirm the match is correct at step 190. Alternatively, if the enquiry at step 185 is answered in the negative, and it is therefore determined that multiple potential matches have been found, processing continues at step 195 where the EDD system presents the user with a list of possible matches to data points from the EHR search from which they can choose to guide the software to identify the correct EHR variable. Thus, in either case, if one or more matches are found, the system will provide the user with an indication in the UI with details of the match(es) between the snippet content and a corresponding field(s) in the EHR. In some cases, the matching of EHR fields and the EDD may be facilitated by the use of generative AI where multiple fields with the same label may be further specified by other parameters of the search such as the visit date, or the like.

Whether processing progresses via step 190 or step 195, at step 200 it is enquired whether the user confirms the match between snippet and a single EHR data point. If this enquiry at step 200 is answered in the negative, and the user therefore is unable to confirm a match between the snippet and a single EHR data point, processing returns to step 180 and continues with the snippet method as described above. If on the other hand, the enquiry at step 200 is answered in the affirmative, and therefore it is determined that the user has confirmed a single match between the snippet and a single EHR data point, processing continues at step 205 where the EDD system obtains the unique field or variable ID from the EHR system via the API and stores this with the CRF question. Processing then continues at step 210 where all future instances of the same CRF question can be automatically populated via API connectivity to the EHR system. The site user can thus opt to permanently establish a link between the CRF question and a unique database variable in the EHR system, allowing future encounters with the same question to be automatically populated into the CRF via the API integration, thus allowing the user to take a SC, and upon clicking the hotspot, skipping the need for creation of a snippet. In these cases, the audit trail will still reflect a processing step that was automatically performed by the system. The audit trail will also record the SC as supporting evidence.

Ultimately, implementation of this embodiment of the present invention would preferably serve to provide pharmaceutical companies and other drug development organizations who run clinical trials with an immediate, low-cost solution to deploy data collection using source images and data extraction, while passively allowing the computer system to learn mappings to an EHR database via an API integration. These mappings may then be saved and re-used for any future clinical trials at a particular clinic using the same EHR system.

Furthermore, by using computer vision techniques, EHR application introspection (e.g. Data Object Model (DOM) inspection for web apps and windows forms introspection for desktop apps), and fingerprinting of EHR API messages, the system is able to identify other sites using the same EHRs and re-use learned mappings for those EHRs from all previous learned API mappings. Similarly, by comparing the results of all previously learned API mappings with EHR CRF data being extracted through manual and automated snippeting processes, viable existing mappings are preferably quickly identified, if any exist, either in their entirety or by synthesizing matching subsets of multiple distinct, previously learned mappings. These automated learnings are preferably augmented and refined through user-interaction whereby users are able to help to resolve ambiguities or unmatched fields and validate mappings by examining captured source documents and inferred CRF+EHR data fields using tools within the system to efficiently visualize, validate, and refine these learned mappings.

It is important to note that in an embodiment of the invention, the system is able to retrospectively correlate EDD snippet information, EHR data, patient identifiers, patient clinic visit date, and FHIR data to match specific API resource elements to EDD snippets and the CRF fields/study variables they represent. This then allows the data collection process to begin immediately and continue while legal, operational, and technical requirements of an API integration (such as FHIR) were addressed with the hospital/clinical trial site administration. In this way, API integrations, which often take some time to implement for each new study site, are no longer blocking dependencies for robust data collection. Study data collection can then begin immediately through the aforementioned source capture, snippeting, OCR, and data entry processes, and in the process, mappings between EHR data and CRF fields for the study would be inherently mapped out.

Once an API integration is implemented for a given site in accordance with the embodiment of the invention, relevant EHR resources for all patients participating in the study would be ingested. Ingested API data for each patient and visit, paired with these robust EHR-CRF mappings built through the snippeting-based data collection that has already been underway, is then preferably automatically analyzed and correlated, using machine learning and other statistical techniques, to build mappings from CRF questions to their corresponding specific EHR resource elements. In this fashion, through normal operation of the previously described snippeting-based data collection combined with EHR API data, a robust mapping of EHR API resource elements to the CRF fields they represent is automatically built, validated, and continually refined.

This can be thought of as a supervised learning process, where user-driven data collection simultaneously trains a machine learning model capable of automatically extracting all of the same data elements from API messages without any user input, ultimately fully automating the extraction of any study data elements from any EHR via an API, importantly, without the traditional, tedious, time-consuming process of manually building these mappings for every EHR involved in a study. Robust API integrations are automatically learned, validated, and continually refined during snippeting data collection without the need for additional human input.

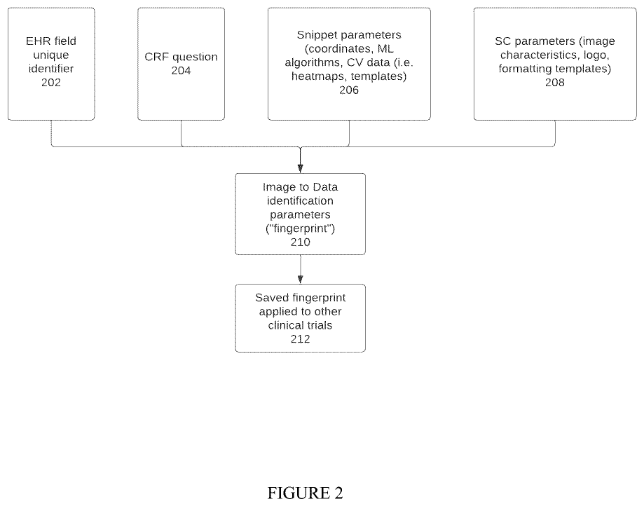

FIG. 2 shows an exemplary flowchart of learned mappings used to extrapolate finding new matches. The unique EHR field identifier 202, CRF question 204, snippet parameters 206 such as coordinates, machine learned algorithms, and/or CV data such as heatmaps, templates, and SC parameters 208, such as image characteristics, logo and/or formatting template are used to create data identification parameters 210 called fingerprints. Fingerprints are then saved 212 and can be applied to other clinical trials. Existing learned mappings can be tested, validated, and applied to new clinical trial sites or EHRs after only a handful of snippeting-based data collections. Once a positive mapping has been achieved for a particular CRF question, the EDD system saves information about the ingredients that were needed to arrive at the matching including the SC, the snippet, the CRF question, the EHR field and any other metainformation involved in the API process. This is considered to be a “fingerprint” that is unique to the data point with parameters used by the EDD System to trace a path to a particular EHR field in reverse, starting with only an image. For example, in the process of one clinical trial, the EDD system would maintain a record of fingerprints for a particular clinical trial site and the EHR system used. In a future unrelated study, the EDD system would recognize characteristics of the SC and identify the EHR platform based upon characteristics such as layout, branding, color schemas, fonts, etc. and the location of the clinical trial site. Previously identified mappings between CRF questions and EHR field identifiers could be immediately tested using API connectivity based upon the snippets created in the EDD system. This is possible using previously learned CRF-EHR mappings from the current study, but also from other studies based on automatically inferred (i.e., using field type, unique database field or variable identifiers, name, keywords, and other contextual metadata) or manually defined mappings of CRF fields across studies. EHR types can also be identified using computer vision and machine learning techniques (i.e., “fingerprinting” based on feature matching, logo identification, etc.) and previously learned mappings for the same EHR can be prioritized for automatic testing and validation against initial human-driven data entry. This approach is analogous to transfer learning, whereby existing learned mappings (i.e., CRF-EHR mapping models) can be evaluated against a small sample of user-driven data entry, and selected for use with a new EHR, either in whole or in part, based on the degree of matching observed in these initial evaluations. In this way, all existing learning can be applied, combined, re-used, and evolved to further streamline the mapping process for new EHRs and new clinical trial sites. Over time, a large-scale library of these learned mappings is built, reducing the degree of human input required to build automated API-based CRF data extraction for each new EHR, clinical site, or study CRF and continually accelerating and improving automated data extraction.

### General

The present invention can take the form of an entirely hardware embodiment, an entirely software embodiment, or an embodiment containing both hardware and software elements. In an exemplary embodiment, the present invention is implemented in software, which includes but is not limited to firmware, resident software, and microcode. Any conventional EMR system can be used as a source of SC, such as Cerner, Epic Systems and Allscripts. The upload of standalone files such as JPEGs, multipage PDF documents or images of paper documents may also be used. These go through the same redaction and OCR process. Preferably, the systems and methods of the present invention are implemented in a client/server network connected via the Internet.

The systems and methods of the present invention will also preferably use data security, encryption, and data capture and transfer protocols that will enhance patient privacy and security and add desirable authentication and verification features. Preferably, all data will be transferred over SSL connections (also known as HTTPS). Preferably, the systems and methods of the invention will use data encryption (public/private key pair) to protect patient medical records represented in SD media, and data encryption will protect both data and media while they are stored and while they are being transferred, ensuring that only the intended recipients are able to access/view them. Preferably, every user must be authenticated on the system by logging in with their private credentials. Preferably, during each interaction with the server, the server confirms the authenticity of the request for interaction by authentication tokens issued by the server. Preferably, the system will require the users to change their passwords periodically. Preferably, users will only have access to the functionality assigned to them by the system administrator. Preferably, information such as patient or subject ID, data capture date, and other necessary identifying information is embedded in the SC and SD image itself as well as included in metadata, and accessible to qualified users and viewers of the EDD. This may also include a unique identifying serial number, subject ID, date/time of capture, IP addresses, user information such as user web browser and device type, for example.

In another preferred embodiment, no media or data is saved to any local machine or device, either by the machine or device as it is created or by the monitor/Sponsor or Investigator when viewing it. Rather, data is captured directly from the screen output by the inventive software and is not handled by the native Operating System, which might write that data to disk, even if only as temporarily cached files. Any additional image processing that may be required, such as file compression for storage, is handled by servers away from the local machine or device. Preferably, the computer systems and programs used in embodiments of the invention do not save the EDD or other files created incident to the operation of the invention as a file that can be recalled at a later time. SC can be obtained from any EMR software running on the same machine as the inventive software, such that the EMR software displays its information on the same screen(s) as are accessible by software implementing the invention. Additional digital media imported by the invention from any external source, such as photographs of paper documents or medical scans, are treated in the same manner as SC once loaded.

Furthermore, the present invention can take the form of a computer program product or products accessible from a computer-usable or computer-readable medium providing program code for use by or in connection with a computer system or any instruction execution system. The computer program product includes the instructions that implement the method of the present invention. A computer-usable or computer readable medium can be any apparatus that can contain, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random-access memory (RAM), a read-only memory (ROM), a rigid magnetic disk, and an optical disk. Current examples of optical disks include compact disk-read only memory (CD-ROM), compact disk-read/write (CD-R/W), and DVD.

A computer system suitable for storing and/or executing program code includes at least one processor coupled directly or indirectly to memory elements through a system bus. The memory elements include local memory employed during actual execution of the program code, bulk storage, and cache memories that provide temporary storage of at least some program code to reduce the number of times code is retrieved from bulk storage during execution. Input/output (I/O) devices (including but not limited to keyboards, displays, pointing devices, etc.) can be coupled to the computer system either directly or through intervening I/O controllers. Network adapters may also be coupled to the computer system in order to enable the computer system to become coupled to other computer systems or remote printers or storage devices through intervening private or public networks. Modems, cable modems, and Ethernet cards are just a few of the currently available types of network adapters. The computer system can also include an operating system and a computer filesystem.
It is to be understood that the above description and examples are intended to be illustrative and not restrictive. Many embodiments will be apparent to those of skill in the art upon reading the above description and examples. The scope of the invention should, therefore, be determined not with reference to the above description and examples but should instead be determined with reference to the appended claims, along with the full scope of equivalents to which such claims are entitled. The disclosures of all articles and references, including patent applications and publications, are incorporated herein by reference for all

What is claimed:

1. A method for mapping a database field having data elements in an electronic health record to a question on a clinical trial case report form (CRF), comprising:
extracting an image of a source document included in the electronic health record and creating one or more snippets, wherein the one or more snippets includes data elements;
selecting one or more of the snippets;
recognizing one or more data elements from the one or more snippets;
inputting one or more of the recognized data elements into an Application Programming Interface (API);
creating one or more search terms using the one or more recognized data elements;
searching an Electronic Health Record (EHR) using the API for one or more search terms;
identifying one or more data elements in the EHR as corresponding to the one or more search terms; and
presenting the one or more identified elements to a user.

2. The method of claim 1, wherein the step of creating the one or more snippets further comprises:
storing the source document image;
determining one or more desired inputs to the Case Report Form (CRF);
identifying one or more hotspots in the source document image comprising key words associated with the one or more desired inputs to the CRF;
confirming the accuracy of the hotspot as an appropriate input to the CRF; and
designating the hotspot as a snippet.

3. The method of claim 2, wherein each of the recognized one or more data elements comprises a predefined label corresponding to the desired input of the CRF.

4. The method of claim 3, further comprising adjusting the hotspot to include a data value associated with the predefined label.

5. The method of claim 4, wherein the adjusting of the hotspot is performed by a user.

6. The method of claim 4, wherein the adjusting of the hotspot is performed automatically by a processor.

7. The method of claim 2, further comprising
confirming a match between the one or more presented identified elements and a corresponding one or more data elements from the snippet.

8. The method of claim 7, further comprising:
extracting one or more identifiers from the EHR that includes the confirmed one or more matched elements; and
storing the one or more identifiers as corresponding to a desired input to the CRF.

9. The method of claim 8, further comprising:
employing the one or more stored identifiers and corresponding CRF input to correlate a desired input from an EHR to a similar desired input to another CRF element.

10. The method of claim 9, further comprising:
retaining the one or more stored identifiers and corresponding CRF input at a remote storage location; and
accessing the retained stored identifiers and corresponding CRF input for use by a second user.

11. The method of claim 10, wherein the second user is located at a remote location from the first user.

12. A computerized system for mapping a database field having data elements in an electronic health record to a question on a clinical trial case report form (CRF), comprising:
at least one processor having a snippet tool extracting an image of a source document included in an electronic health record and creating one or more snippets, wherein the one or more snippets includes data elements;
an Application Programming Interface (API), wherein one or more of the data elements are input into the Application Programming Interface (API) as a search term;
the at least one processor searching an Electronic Health Record (EHR) using the API for one or more search terms and identifying one or more data elements in the EHR as corresponding to the one or more search terms and presenting the one or more identified elements to a user.

13. The system of claim 12, wherein the snippet tool is configured to store a source document image;
determine one or more desired inputs to the Case Report Form (CRF);
identify one or more hotspots in the source document image comprising key words associated with the one or more desired inputs to the CRF;
confirm the accuracy of the hotspot as an appropriate input to the CRF; and
designate the hotspot as a snippet.

14. The system of claim 13, wherein the recognized one or more data elements comprises a predefined label corresponding to the desired input of the CRF.

15. The system of claim 14, further comprising a hotspot tool configured to adjust the hotspot to include a data value associated with the predefined label.

16. The method of claim 15, wherein the adjusting of the hotspot is performed by a user.

17. The method of claim 15, wherein the at least one processor automatically adjusts the hotspot.

18. The system of claim 13, further comprising a matching tool confirming a match between the one or more presented identified elements and a corresponding one or more data elements from the snippet.

19. The system of claim 18, wherein the matching tool extracts one or more identifiers from the EHR that includes the confirmed one or more matched elements and stores the one or more identifiers as corresponding to a desired input to the CRF.

20. The system of claim 19, wherein the matching tool correlates the one or more stored identifiers and corresponding CRF input to a desired input from the EHR to a similar desired input to another CRF element.

21. The system of claim 20, further comprising a remote storage location, wherein the at least one processor retains the one or more stored identifiers and corresponding CRF input at the remote storage location, wherein a second user accesses the retained stored identifiers and corresponding CRF input.

22. The system of claim 21, wherein the second user is located at a remote location from the first user.

23. A computer processor configured to implement a method for mapping a database field having data elements in an electronic health record to a question on a clinical trial case report form (CRF), comprising:
extracting an image of a source document included in the electronic health record and creating one or more snippets, wherein the one or more snippets includes data elements;
selecting one or more of the snippets;
recognizing one or more data elements from the one or more snippets;
inputting one or more of the recognized data elements into an Application Programming Interface (API);
creating one or more search terms using the one or more recognized data elements;
searching an Electronic Health Record (EHR) using the API for one or more search terms;
identifying one or more data elements in the EHR as corresponding to the one or more search terms; and
presenting the one or more identified elements to a user.
